(19) United States
(12) Patent Application Publication
(10) Pub. No.: US 2012/0301898 A1
(43) Pub. Date: Nov. 29, 2012

(54) ASSESSING NON-ALCOHOLIC FATTY LIVER DISEASE

(76) Inventor: Michael R. Charlton, Rochester, MN (US)

(21) Appl. No.: 13/584,535

(22) Filed: Aug. 13, 2012

Related U.S. Application Data
(62) Division of application No. 12/280,502, filed on Jan. 28, 2009.
(60) Provisional application No. 60/777,964, filed on Mar. 1, 2006.

(51) Int. Cl. G01N 33/53

(52) U.S. Cl. 435/7.9; 436/501

(57) ABSTRACT

The document provides methods and materials related to assessing NAFLD in a mammal. For example, methods and materials for determining whether or not a mammal has an NAFLD are provided. In addition, methods and materials for determining whether a mammal with an NAFLD has a severe or mild form of the NAFLD as well as methods and materials for determining whether a mammal with an NAFLD is likely to experience a severe or mild form of the NAFLD are provided.

### CROSS-REFERENCE TO RELATED APPLICATIONS

This application is a divisional application of U.S. application Ser. No. 12/280,502, filed Jan. 28, 2009, which is a National Stage application under 35 U.S.C. §371 of International Application No. PCT/US2007/063101 having an International Filing Date of Mar. 1, 2007, which claims the benefit of U.S. Provisional Application Ser. No. 60/777,964, having a filing date of Mar. 1, 2006. The disclosures of the prior applications are considered part of (and are incorporated by reference in) the disclosure of this application.

### BACKGROUND

1. Technical Field

This document relates to methods and materials involved in assessing non-alcoholic fatty liver disease (NAFLD). For example, this document provides methods and materials for determining whether or not a mammal has severe non-alcoholic steatohepatitis (NASH).

2. Background Information

NAFLD includes a spectrum of histological findings, ranging from simple steatosis to NASH with progressive fibrosis and liver failure. In addition, NAFLD is recognized as an important and increasingly common cause of cirrhosis and liver failure (Wanless and Lentz, Hepatology, 12:1106-1110 (1990); Charlton, Clin. Gastro. Hepat., 2(12):1048-58 (2004); Kim et al., Transplantation, 62:1802-1805 (1996); and Charlton et al., Liver Transplantation, 7:608-614 (2001)). The prevalence of cirrhosis among obese patients with NASH has been reported to be between 8-13.8 percent (Wanless and Lentz, Hepatology, 12:1106-1110 (1990) and Pinto et al., Dig. Dis. Sci., 41:172-179 (1996)). The biological basis of the histological diversity of severity of NAFLD (i.e. why some patients develop simple steatosis and others NASH with advanced fibrosis) is not known. Identifying patients at risk for the development of NASH with progressive fibrosis is of central importance to mitigating the impact of NAFLD as a whole.

### SUMMARY

This document involves methods and materials related to assessing NAFLD in a mammal. For example, this document provides methods and materials for determining whether or not a mammal (e.g., a human) has a mild or severe NAFLD. In some cases, the methods and materials provided herein can be used to determine whether or not a mammal has severe non-alcoholic steatohepatitis (NASH). Identifying mammals as having either mild or severe NAFLD using the methods and materials provided herein can allow clinicians to select treatment options at an early stage of an NAFLD. In addition, the methods and materials provided herein can be used to identify mammals as having either mild or severe NAFLD without the need for an invasive liver biopsy procedure.

As described herein, dehydroepiandrosterone (DHEA) levels can be measured and used to assess the mildness or severity of an NAFLD (e.g., the histological mildness or severity of an NAFLD). For example, human patients with a low plasma level of DHEA (e.g., less than 0.45 μg of DHEA per mL blood) can be classified as being at increased risk of having a severe form of NAFLD such as severe NASH with more advanced liver fibrosis, while human patients with a high plasma level of DHEA (e.g., greater than 0.45 μg of DHEA-S, the sulfated form of DHEA, per mL of blood) are highly unlikely to have a severe form of NAFLD. In some cases, circulating DHEA levels can be used to identify mammals (e.g., humans) at risk for developing NASH with progressive fibrosis.

In general, one aspect of this document features a method for assessing a mammal for an NAFLD. The method comprises, or consists essentially of, determining whether or not a mammal contains a reduced level of DHEA or a DHEA related compound, wherein the presence of the reduced level indicates that the mammal has an NAFLD. The mammal can be a human. The NAFLD can be NASH with progressive fibrosis. The determining step can comprise, or consist essentially of, determining the level of DHEA or the DHEA related compound in a blood sample from the mammal. The reduced level can be less than 0.45 μg of DHEA or DHEA-S per mL of blood. The method can comprise, or consist essentially of, determining whether or not the mammal contains the reduced level of DHEA. The method can comprise, or consist essentially of, determining whether or not the mammal contains the reduced level of the DHEA related compound. The DHEA related compound can be DHEA-S.

In another embodiment, this document features a method for determining whether or not a mammal having an NAFLD has severe NAFLD. The method comprises, or consists essentially of, determining whether or not the mammal contains a reduced level of DHEA or a DHEA related compound, wherein the presence of the reduced level indicates that the mammal has the severe NAFLD. The mammal can be a human. The severe NAFLD can be NASH with progressive fibrosis. The determining step can comprise, or consist essentially of, determining the level of DHEA or the DHEA related compound in a blood sample from the mammal. The reduced level can be less than 0.45 μg of DHEA or DHEA-S per mL of blood. The method can comprise, or consist essentially of, determining whether or not the mammal contains the reduced level of DHEA. The method can comprise, or consist essentially of, determining whether or not the mammal contains the reduced level of the DHEA related compound. The DHEA related compound is DHEA-S. The absence of the reduced level can indicate that the mammal does not have the severe NAFLD.

In another embodiment, this document features a method for determining whether or not a mammal having an NAFLD has mild NAFLD. The method comprises, or consists essentially of, determining whether or not the mammal contains a reduced level of DHEA or a DHEA related compound, wherein the absence of the reduced level indicates that the mammal has the mild NAFLD. The mammal can be a human. The mild NAFLD can be steatosis. The determining step can comprise, or consist essentially of, determining the level of DHEA or the DHEA related compound in a blood sample from the mammal The reduced level can be less than 0.45 μg of DHEA or DHEA-S per mL of blood. The method can comprise, or consist essentially of, determining whether or not the mammal contains the reduced level of DHEA. The method can comprise, or consist essentially of, determining whether or not the mammal contains the reduced level of the DHEA related compound. The DHEA related compound can be DHEA-S. The presence of the reduced level can indicate that the mammal has severe NAFLD.

In another embodiment, this document features a method for determining whether or not a mammal having an NAFLD is likely to develop severe NAFLD. The method comprises, or consists essentially of, determining whether or not the mammal contains a reduced level of DHEA or a DHEA related compound, wherein the presence of the reduced level indicates that the mammal is likely to develop the severe NAFLD. The mammal can be a human. The severe NAFLD can be NASH with progressive fibrosis. The determining step can comprise, or consist essentially of, determining the level of DHEA or the DHEA related compound in a blood sample from the mammal. The reduced level can be less than 0.45 μg of DHEA or DHEA-S per mL of blood. The method can comprise, or consist essentially of, determining whether or not the mammal contains the reduced level of DHEA. The method can comprise, or consist essentially of, determining whether or not the mammal contains the reduced level of the DHEA related compound. The DHEA related compound can be DHEA-S. The absence of the reduced level can indicate that the mammal does not have the severe NAFLD.

In another embodiment, this document features a method for determining whether or not a mammal having an NAFLD is likely to develop mild NAFLD. The method comprises, or consists essentially of, determining whether or not the mammal contains a reduced level of DHEA or a DHEA related compound, wherein the absence of the reduced level indicates that the mammal is likely to develop the mild NAFLD. The mammal can be a human. The mild NAFLD can be steatosis. The determining step can comprise, or consist essentially of, determining the level of DHEA or the DHEA related compound in a blood sample from the mammal. The reduced level can be less than 0.45 μg of DHEA or DHEA-S per mL of blood. The method can comprise, or consist essentially of, determining whether or not the mammal contains the reduced level of DHEA. The method can comprise, or consist essentially of, determining whether or not the mammal contains the reduced level of the DHEA related compound. The DHEA related compound can be DHEA-S. The presence of the reduced level can indicate that the mammal is likely to develop severe NAFLD.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting DHEA levels (μg/mL) for human patients with either stage 0-2 or stage 3-4 fibrosis.

FIG. 2 is a graph plotting DHEA levels (μg/mL) for human patients with stage 0, 1, 2, 3, or 4 fibrosis. The diamonds indicate the mean and standard errors.

FIG. 3 is a graph plotting DHEA levels for patients with human chronic cholestatic liver disease (e.g., primary biliary cirrhosis or primary sclerosing cholangitis with stage 0, 1, 2, 3, or 4 fibrosis).

FIG. 4 is a graph plotting sensitivity versus specificity of using DHEA levels to distinguish between patients with and without severe NAFLD.

FIG. 5 is a graph plotting age versus fibrosis stage.

### DETAILED DESCRIPTION

The document provides methods and materials related to assessing NAFLD in a mammal (e.g., human, dog, cat, horse, cow, goat, pig, and rodent). For example, this document provides methods and materials for determining whether or not a mammal has an NAFLD. Examples of NAFLD include, without limitation, simple steatosis, steatohepatitis, and steatohepatitis with increased hepatic fibrosis. An NAFLD can be mild to severe and can be associated with obesity and insulin resistance. This document also provides methods and materials for determining whether a mammal with an NAFLD has a severe or mild form of NAFLD. In addition, this document provides methods and materials for determining whether a mammal with NAFLD is likely to experience a severe or mild form of NAFLD. Severe NAFLD included, without limitation, NASH with progressive fibrosis, which can be determined by the presence of bridging fibrosis or cirrhosis.

As described herein, the level of DHEA, or a DHEA related compound such as DHEA-S (the sulfate form of DHEA), the precursors of DHEA, or the metabolites of DHEA, within a mammal can be determined and used to assess whether or not a mammal has an NAFLD. For example, if the level of DHEA or a DHEA related compound in a sample from a mammal is a reduced level, then the mammal can be classified as having an NAFLD. The level of DHEA or a DHEA related compound can be used to determine whether a mammal with NAFLD has a mild or severe form of NAFLD. For example, if the level of DHEA or a DHEA related compound in a sample is not a reduced level, then the mammal can be classified as having a mild form of the NAFLD. In some cases, the level of DHEA or a DHEA related compound can be used to determine whether a mammal with NAFLD will develop a mild or severe form of NAFLD. For example, if the level of DHEA or a DHEA related compound in a sample from a mammal with NAFLD is a reduced level, then the mammal can be classified as being likely to develop a severe form of NAFLD. If the level of DHEA or a DHEA related compound in a sample is not a reduced level, then the mammal can be classified as being likely to develop a mild form of NAFLD.

The term “reduced level” as used herein with respect to the level of DHEA is any level of DHEA that is less than a reference level for DHEA adjusted for gender and age. The term “reference level” as used herein with respect to DHEA is the level of DHEA typically found in healthy control mammals. For example, a reference level of DHEA can be the average level of DHEA that is present in samples obtained from a random sampling of healthy control mammals (e.g., 5, 10, 25, or 50 healthy control mammals). In the case of humans, a reduced level of DHEA can be any level less than 1.0 μg/mL, 0.75 μg/mL, 0.5 μg/mL, 0.45 μg/mL, 0.4 μg/mL, 0.35 μg/mL, 0.3 μg/mL, 0.25 μg/mL, or 0.2 μg/mL. For example, a reduced level of DHEA for a human can be between 1.0 μg/mL and 0.01 μg/mL. (e.g., between 0.75 μg/mL and 0.01 μg/mL; between 0.5 μg/mL and 0.01 μg/mL; or between 0.45 μg/mL and 0.01 μg/mL).

The term “reduced level” as used herein with respect to the level of a DHEA related compound is any level of a DHEA related compound that is less than a reference level for a DHEA related compound adjusted for gender and age. The term “reference level” as used herein with respect to a DHEA related compound is the level of a DHEA related compound typically found in healthy control mammals. For example, a reference level of a DHEA related compound can be the average level of the DHEA related compound that is present in samples obtained from a random sampling of healthy control mammals (e.g., 5, 10, 25, or 50 healthy control mammals). In the case of humans, a reduced level of DHEA-S can be any level less than 1.0 μg/mL, 0.75 μg/mL, 0.5 μg/mL, 0.45 μg/mL, 0.4 μg/mL, 0.35 μg/mL, 0.3 μg/mL, 0.25 μg/mL, or 0.2 μg/mL. For example, a reduced level of DHEA-S for a human can be between 1.0 μg/mL and 0.01 μg/mL. (e.g., between 0.75 μg/mL and 0.01 μg/mL; between 0.5 μg/mL and 0.01 μg/mL; or between 0.45 μg/mL and 0.01 μg/mL.

It will be appreciated that levels from comparable samples are used when determining whether or not a particular level is a reduced level or not. For example, the average level of DHEA present in blood from a random sampling of healthy humans can be 4 μg/mL of blood, while the average level of DHEA present in liver tissue samples from the same random sampling of healthy humans can be 3 μg/g of tissue. In this case, the reference level for DHEA in blood would be 4 μg/mL of blood, and the reference level for DHEA in liver tissue would be 3 μg/g of liver tissue.

A reduced level of a DHEA or DHEA related compound can be any level provided that the level is less than a corresponding reference level for the DHEA or DHEA related compound. For example, a reduced level of DHEA or a DHEA related compound can be 1, 2.5, 5, 10, 20, 30, 50, 75, 90, or more percent less than the reference level for the DHEA or DHEA related compound. In addition, a reference level can be any amount. For example, a reference level for DHEA can be 2, 3, 5, 6, 7, 8, or more μg/mL of blood.

Any method can be used to determine the level of DHEA or a DHEA related compound present within a sample. For example, radioimmunoassays (e.g., commercial radioimmunoassay) and enzyme immunoassays can be used to determine the level of DHEA present within a sample. Any type of sample can be used to evaluate the level of DHEA or a DHEA related compound including, without limitation, blood, serum, or tissue (e.g., liver tissue) samples. Any method can be used to obtain a sample. For example, a blood sample can be obtained using a needle or catheter and standard blood drawing techniques, while a tissue sample can be obtained by biopsy. Once obtained, a sample can be manipulated prior to measuring the level of DHEA or a DHEA related compound.

This document also provides methods and materials to assist medical or research professionals in determining whether or not a mammal has an NAFLD, whether a mammal with NAFLD has mild or severe NAFLD, or whether a mammal with NAFLD is likely to develop mild or severe NAFLD. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of a DHEA or DHEA related compound in a sample, and (2) communicating information about the determined level to that professional. Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Markers for NAFLD

### Study Design

The following three groups of participants (total participants=128) were used in this study: (1) patients with liver biopsy demonstrating simple steatosis or NASH with fibrosis stage 0-2 (mild group, n=53); (2) patients with liver biopsy demonstrating NASH with more advanced fibrosis (severe group, n=25); and (3) patients with other liver diseases (e.g., primary biliary cirrhosis and primary sclerosing cholangitis, n=44). The participants with NAFLD (mild and severe NAFLD groups) were recruited from patients seen at an hepatobiliary clinic. Serum samples were obtained prospectively at the same time as liver biopsies from participating patients who were scheduled to undergo liver biopsy for investigation of suspected liver disease (NAFLD or cholestatic liver disease). Patients with NAFLD who had secondary causes of steatohepatitis (e.g., drugs or gastric surgery for obesity) and patients with other etiologies of chronic liver disease (e.g., excessive alcohol consumption, viral hepatitis B or C, cholestatic liver disease, hemochromatosis, Wilson's disease, drug induced liver disease, and alpha 1-antitrypsin deficiency) were excluded from this study.

Primary biliary cirrhosis was diagnosed based upon an AMA of 1:40, biochemical abnormalities, and a liver biopsy demonstrating histological features of PBC (portal hepatitis with granulomatous destruction of the bile ducts). Stage 2 is characterized by periportal hepatitis and bile duct proliferation. The presence of fibrous septa or bridging necrosis is classified as stage 3, and cirrhosis is classified as stage 4 (Villareal and Holloszy, JAMA, 292(18):2243-8 (2004)). The presence of fibrosis or cirrhosis indicated a worse prognosis than if no fibrosis was seen on biopsy. Primary sclerosing cholangitis was diagnosed by the findings of multifocal structuring and dilation of intrahepatic and/or extrahepatic bile ducts on cholangiography, biochemical abnormalities, and the presence of histological finding of fibrous obliteration of small bile ducts.

A detailed alcohol consumption history was obtained for all participants. All participants drank less than 20 g or ethanol per day. Diagnosis of diabetes mellitus was based on the American Diabetes Association or World Health Organization criteria.

### Liver Histology in NAFLD

An experienced liver pathologist, who was blinded to the clinical data, reviewed the liver biopsy specimens. Patients with NAFLD were divided into mild (simple steatosis (SS) and NASH with fibrosis stage 0-2) and severe (NASH with fibrosis stage 3-4). NASH was also graded for inflammatory activity as follows: mild (grade 1) and moderate/severe (grades 2 and 3). The fibrosis staging system was classified as follows: 0=no fibrosis; stage 1=zone 3 predominant pericellular fibrosis; stage 2=zone 3 fibrosis plus periportal fibrosis; stage 3=bridging fibrosis; and stage 4=cirrhosis. Scoring of steatosis included both micro- and macro-vesicular steatosis and was based on the percentage area of the parenchyma that was fatty. Less than 33% indicated the presence of mild steatosis; between 33% to 65% indicated moderate steatosis; and greater than 66% indicated severe steatosis.

### Adipokines and DHEA and Glucose

Leptin, resistin, adiponectin, and DHEA-S concentrations in blood drawn at the time of the liver biopsies were measured by radioimmunoassays using a commercial kit (Diagnostic Systems Lab. Webster, Tex.). Plasma glucose concentrations were measured enzymatically with an auto analyzer (Beckman Instruments, Fullerton, Calif.).

### Patient Characteristics

Each patient's age, gender, body mass index (BMI), aspartate aminotransferase (AST) levels, alanine aminotransferase (ALT) levels, AST/ALT ratio, total bilirubin levels, fasting glucose levels, triglyceride levels, cholesterol levels, glycosylated hemoglobin levels, creatinine levels, and hematological profile were recorded. In addition, a detailed alcohol consumption history, smoking history, and diagnosis of diabetes were recorded for each patient.

### Statistical Methods

Continuous variables were summarized with means and standard deviations, while categorical variables with frequencies and percentages. Continuous data were analyzed using Analysis of Variance if normally distributed, or the non-parametric Wilcoxon Rank Sums test if non-normally distributed. The chi-squared test or Fisher's exact test (where appropriate) was used for comparison of frequency data. A simple logistic regression models was fit to correlate DHEA-S levels in patients with mild vs. severe NAFLD. The area under the receiver operating characteristics (ROC) curve was calculated and adjusted for factors that influence circulating levels of DHEA-S (i.e., age and gender). The accuracy of DHEA-S levels in separating patients with mild and severe NAFLD was determined by calculating sensitivity, specificity, and positive and negative predictive values.

### Results

A large group of indices was assessed for the ability to identify patients with NAFLD and to predict the severity of an NAFLD.

### Study Population

A total of 78 evaluable participants with NAFLD and 44 controls (patients with cholestatic liver disease—CLD) were studied. Clinical, biochemical, and histological stage of disease in participants with NAFLD and controls with chronic cholestatic liver disease were summarized (Table 1). Components of the metabolic syndrome including increased BMI (and thus overweight and obesity), type 2 diabetes, hypertension, and low HDL-cholesterol were significantly more common in patients with NAFLD than in participants with CLD. Participants with CLD had significantly higher levels of aminotransferases, bilirubin, alkaline phosphatase, and total cholesterol, and lower albumin levels when compared to NAFLD patients.

TABLE 1
Clinical and laboratory data of patients with NAFLD
and controls with chronic cholestatic liver disease.

| | NAFLD (n = 78) | CLD (n = 44) | P-value |
|---|---|---|---|
| Age (years) | 50.0 ± 11.1 | 47.1 ± 11.7 | 0.2 |
| Gender (female/male) | 50/28 | 31/13 | 0.6 |
| Race (White) | 76 (97%) | 44 (100%) | 0.6 |
| Body mass index (kg/m²) | 33.0 ± 7.4 | 26.0 ± 6.2 | <0.0001 |
| Overweight | 22 (28%) | 10 (23%) | <0.0001 |
| (BMI 25-30 kg/m²) | | | |
| Obesity | 51 (65%) | 10 (23%) | <0.0001 |
| (BMI >30 kg/m²) | | | |
| Type 2 diabetes mellitus | 32 (41%) | 5 (14%) | 0.003 |
| Hypertension | 39 (50%) | 10 (27%) | 0.03 |
| Hyperlipidemia | 57 (73%) | 27 (73%) | 1.0 |
| History of smoking | | | |
| None | 52 (67%) | 31 (84%) | 0.001 |
| Ex-smoking | 26 (33%) | 3 (8%) | |
| Current smoking | — | 3 (8%) | |
| DHEA-S (μg/mL) | 0.8 ± 1.0 | 0.8 ± 0.7 | 0.9 |
| Fasting glucose (mg/dL) | 116.4 ± 41.8 | 106.5 ± 46.2 | 0.3 |
| Cholesterol (mg/dL) | 194.6 ± 43.0 | 235.1 ± 58.7 | <0.0001 |
| Triglyceride (mg/dL) | 177.7 ± 86.0 | 152.1 ± 86.5 | 0.1 |
| HDL-C (mg/dL) | 42.8 ± 10.2 | 61.8 ± 22.0 | <0.0001 |
| AST (U/L) | 60.0 ± 44.2 | 88.9 ± 62.0 | 0.008 |
| ALT (U/L) | 85.1 ± 88.0 | 181.1 ± 114.4 | 0.04 |
| AST/ALT ratio | 0.98 ± 0.97 | 0.71 ± 0.22 | 0.05 |
| Alkaline phosphatase | 210.5 ± 103.4 | 914.3 ± 737.9 | <0.0001 |
| (U/L) | | | |
| Total bilirubin (mg/dL) | 0.9 ± 0.5 | 2.5 ± 3.4 | 0.004 |
| Albumin (g/dL) | 4.2 ± 0.4 | 3.9 ± 0.5 | 0.001 |
| PT (sec) | 9.8 ± 0.7 | 10.5 ± 2.9 | 0.1 |
| Creatinine (mg/dL) | 1.0 ± 0.2 | 1.0 ± 0.3 | 0.6 |
| Hemoglobin (g/dL) | 13.7 ± 1.3 | 13.1 ± 1.3 | 0.03 |
| Leukocyte count | 5,804.7 ± 1,701.2 | 5,908.1 ± 2,451.3 | 0.8 |
| (cell/mL) | | | |
| Platelet (×10³/mL) | 202.2 ± 68.7 | 190.6 ± 96.6 | 0.5 |

Data are the mean ± SD.
Abbreviation: CLD, cholestatic liver disease (PBC and PSC); HDL-C, high density lipoprotein cholesterol; AST, alanine aminotransferase; ALT, aspartate aminotransferase; PT, prothrombin time.

Histological characteristics of participants with NAFLD were determined (Table 2). All participants with NAFLD had steatosis of varying severity. The great majority (73 out of the 78; 94%) of participants had some degree of lobular inflammation. Severe liver fibrosis (stage 3-4) was present in 25 (32%) patients; 21 (27%) patients did not have fibrosis; and 32 (41%) patients had mild to moderate (stage 1-2) fibrosis.

TABLE 2
Histological findings in patients with NAFLD (n = 78).

| Histologic feature | Number (%) |
|---|---|
| Steatosis | |
| Mild | 36 (46) |
| Moderate | 33 (42) |
| Severe | 9 (12) |
| Lobular inflammation | |
| None | 5 (6) |
| Mild | 48 (62) |
| Moderate | 24 (31) |
| Severe | 1 (1) |
| Fibrosis | |
| None | 21 (27) |
| Stage 1 | 13 (17) |
| Stage 2 | 19 (24) |
| Stage 3 | 13 (17) |
| Stage 4 | 12 (15) |

Results obtained from participants with mild and severe NAFLD were compared (Table 3). Mild and severe NAFLD groups had similar proportions of men and women. Participants in the severe NAFLD group were significantly older, and were more commonly obese and diabetic as compared to participants with mild NAFLD. Participants with severe NAFLD had significantly lower levels of ALT, a higher AST/ALT ratio, lower albumin and hemoglobin levels, and lower platelet counts. Participants in the severe NAFLD group had significantly lower levels of DHEA-S, and higher levels of resistin and CRP. There was a trend for higher levels of leptin among participants with severe NAFLD (p=0.06), whereas adiponectin levels were similar between the two groups.

TABLE 3
Clinical and laboratory data in patients with mild
NAFLD (e.g., simple steatosis and fibrosis stage 0-2)
and severe NAFLD (e.g., fibrosis stage 3-4).

| | Mild NAFLD | Severe NAFLD | |
|---|---|---|---|
| | (n = 53) | (n = 25) | P-value |
| Age (years) | 47.3 ± 11.8 | 55.9 ± 6.4 | 0.0007 |
| Gender (female/male) | 32/21 | 18/7 | 0.3 |
| Race (White) | 51 (96%) | 25 (100%) | 0.6 |
| Body mass index (kg/m²) | 31.8 ± 6.8 | 35.4 ± 8.2 | 0.05 |
| Obesity (BMI >30 kg/m²) | 31 (58%) | 20 (80%) | 0.06 |
| Type 2 diabetes mellitus | 17 (32%) | 15 (60%) | 0.02 |
| Hypertension | 24 (45%) | 15 (60%) | 0.2 |
| Hyperlipidemia | 41 (77%) | 16 (64%) | 0.2 |
| History of smoking | 19 (36%) | 7 (28%) | 0.5 |
| Fasting glucose (mg/dL) | 117.1 ± 48.2 | 114.7 ± 23.9 | 0.3 |
| Cholesterol (mg/dL) | 199.8 ± 46.2 | 183.4 ± 33.1 | 0.2 |
| Triglyceride (mg/dL) | 186.2 ± 92.0 | 159.6 ± 70.1 | 0.2 |
| HDL-C (mg/dL) | 43.0 ± 10.4 | 42.2 ± 10.2 | 0.7 |
| AST (U/L) | 56.8 ± 38.3 | 67.0 ± 54.7 | 0.9 |
| ALT (U/L) | 90.9 ± 92.4 | 73.4 ± 78.6 | 0.04 |
| AST/ALT ratio | 0.8 ± 0.4 | 1.4 ± 1.5 | <0.0001 |
| Alkaline phosphatase (U/L) | 207.1 ± 112.7 | 217.6 ± 81.9 | 0.5 |
| Total bilirubin (mg/dL) | 0.8 ± 0.4 | 1.0 ± 0.7 | 0.07 |
| Albumin (g/dL) | 4.3 ± 0.4 | 4.0 ± 0.3 | 0.004 |
| PT (sec) | 9.7 ± 0.7 | 9.9 ± 0.7 | 0.08 |
| Creatinine (mg/dL) | 1.1 ± 0.2 | 1.0 ± 0.2 | 0.9 |
| Hemoglobin (g/dL) | 14.0 ± 1.1 | 13.2 ± 1.5 | 0.01 |
| Platelet (×10³/mL) | 222.6 ± 66.8 | 158.9 ± 51.2 | <0.0001 |
| DHEA (μg/mL) | 1.1 ± 1.1 | 0.25 ± 0.1 | <0.0001 |
| Leptin (ng/mL) | 24.5 ± 15.2 | 33.9 ± 20.6 | 0.06 |
| Adiponectin (μg/mL) | 20.3 ± 10.6 | 21.6 ± 10.5 | 0.4 |
| Resistin (ng/mL) | 1.7 ± 0.6 | 2.4 ± 0.8 | 0.0008 |
| CRP (mg/L) | 0.4 ± 0.4 | 0.7 ± 0.7 | <0.05 |

Data are the mean ± SD.
Abbreviation: HDL-C, high density lipoprotein cholesterol; AST, alanine aminotransferase; ALT, aspartate aminotransferase; PT, prothrombin time; DHEA, dehydroepiandrosterone; CRP, C-reactive protein.

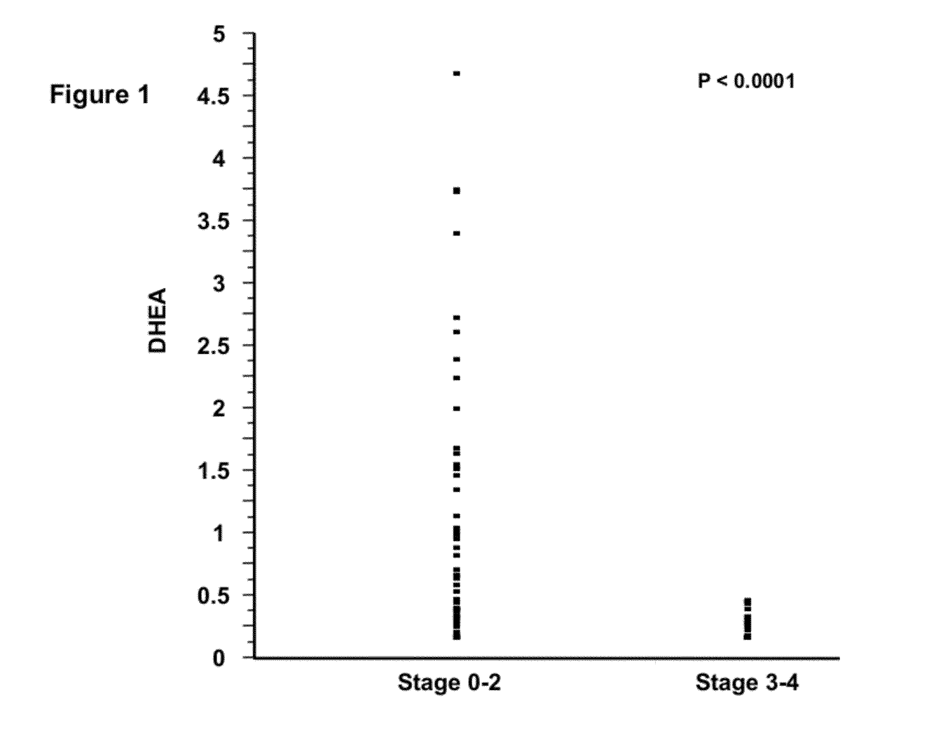

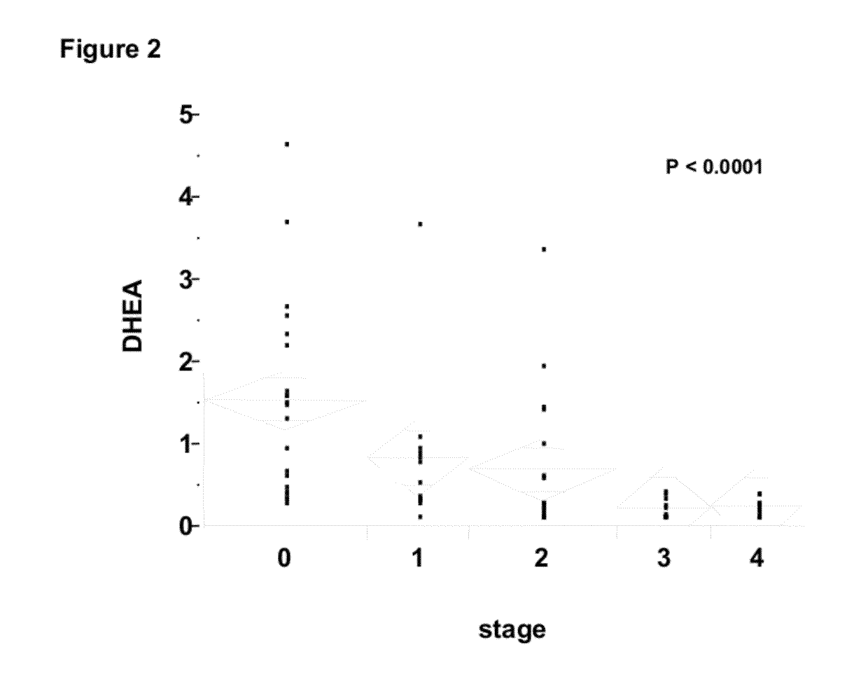

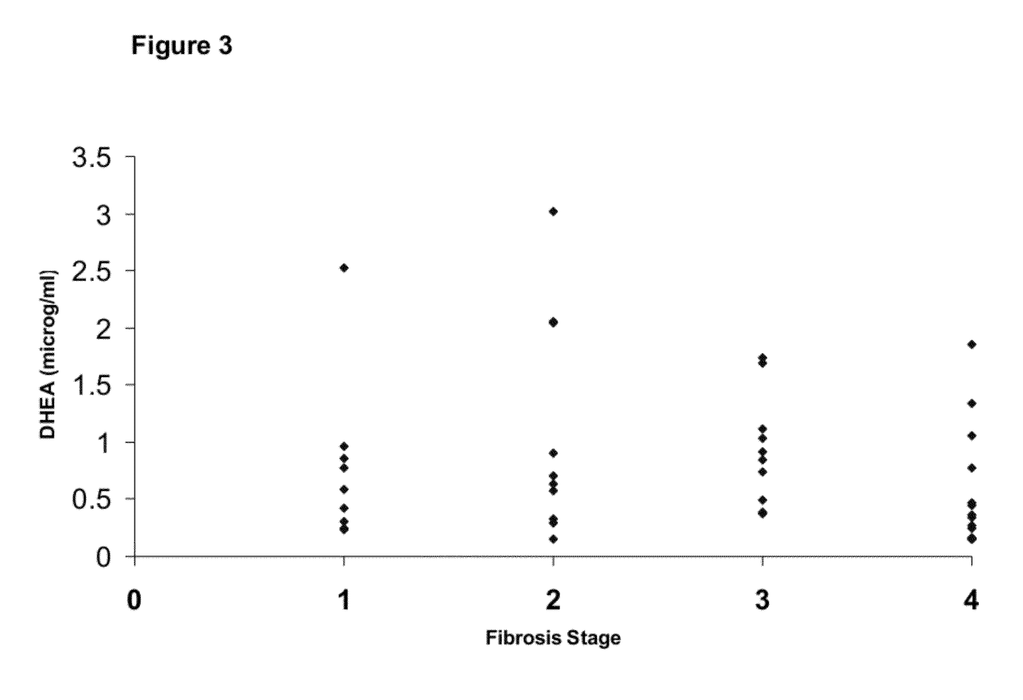

Levels of DHEA were not significantly different between participants with NAFLD and participants with cholestatic liver disease (p=0.9; Table 1). In participants with NAFLD, however, levels of DHEA strongly correlated with histological severity of disease. Participants with NASH+stage 3-4 fibrosis (a severe NAFLD) had significantly lower levels of DHEA as compared to participants with mild NAFLD (FIG. 1). None of the participants with severe NAFLD had levels of DHEA-S above 0.45 μg/mL. A DHEA-S level of greater than 0.45 μg/mL alone had both sensitivity and negative predictive value of 100% in distinguishing between mild and severe NAFLD. The specificity and positive predictive value were 58.5% and 48%, respectively. This indicates that a DHEA-S level above 0.45 μg/mL rules out presence of severe NAFLD with a high degree of accuracy. A “dose effect” of lower DHEA-S levels and fibrosis stage was observed, with mean DHEA levels decreasing with step wise increases in fibrosis stage (FIG. 2). Conversely, levels of DHEA-S did not correlate significantly with severity of the liver disease in participants with chronic cholestasis (FIG. 3).

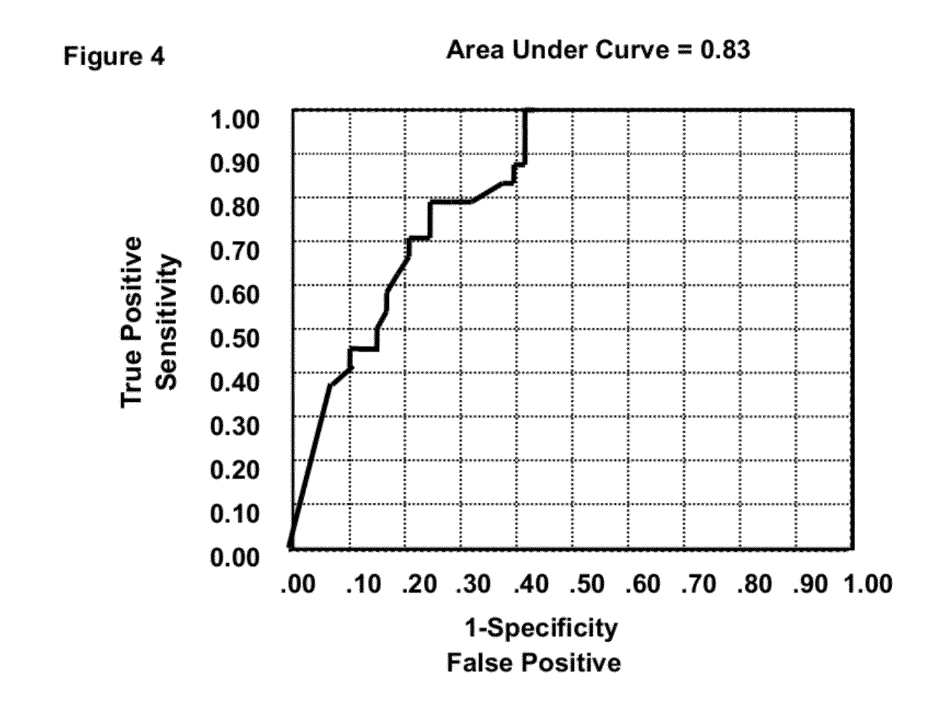

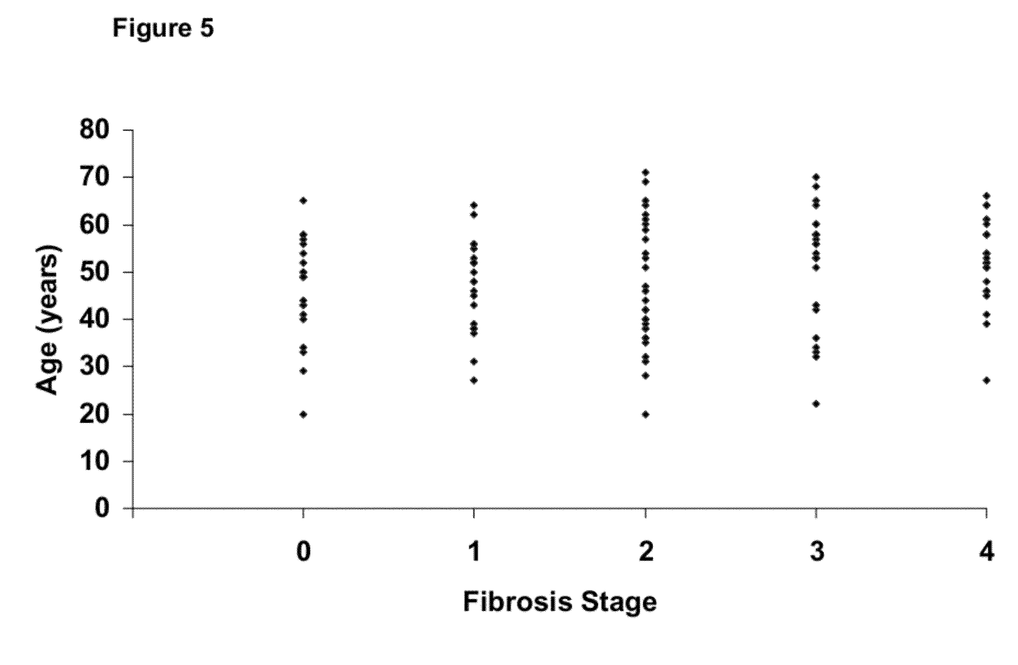

The area under the ROC curve for DHEA-S in separating patients with and without significant fibrosis was 0.83 (FIG. 4). As levels of DHEA-S are different between men and women and lower in older individuals, the area under the ROC curve was adjusted for age and gender. Adjustment for age and gender resulted in a minor increase in the area under the ROC curve to a value of 0.84. Almost all of the predictivity could thus be attributed to DHEA-S levels independent of age and gender. Variation of fibrosis stage with age for NAFLD participants is shown in FIG. 5.

DHEA and DHEA-S are abundant circulating steroid hormones and are produced primarily by the zona reticularis of the adrenal cortex in response to adrenocorticotropic hormone. In humans, DHEA and DHEA-S levels peak at about age 25 years, and decrease progressively thereafter, falling to 5 percent of peak levels by the ninth decade. Since DHEA levels decline with age, it is possible for the lower DHEA levels observed in patients with severe NAFLD to be simply a surrogate of older age and thus duration of disease. Mean age was greater in patients with severe NAFLD when compared to those with mild NAFLD (55.9+6.4 vs. 47.3+11.8 years, P=0.007). As expected, DHEA levels did decline with age, with a wide range of values seen for specific ages. Age, however, was very poorly predictive of severity of NAFLD (FIG. 5).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal having an NAFLD as having severe stage 3 or stage 4 liver fibrosis, wherein said method comprises:
(a) detecting the presence of a reduced level of DHEA or DHEA-S within a blood sample of said mammal by performing an immunoassay, wherein said reduced level is less than 0.45 μg of said DHEA or said DHEA-S per mL of blood, and
(b) classifying said mammal as having said severe stage 3 or stage 4 liver fibrosis.

2. The method of claim 1, wherein said mammal is a human.

3. The method of claim 1, wherein said severe stage 3 or stage 4 liver fibrosis is severe stage 4 liver fibrosis.

4. The method of claim 1, wherein said method comprises detecting the presence of said reduced level of said DHEA within said blood sample.

5. The method of claim 1, wherein said method comprises detecting the presence of said reduced level of said DHEA-S within said blood sample.
